(19) United States
(12) Patent Application Publication
(10) Pub. No.: US 2022/0178923 A1
(43) Pub. Date: Jun. 9, 2022

(54) METHOD FOR DETECTION OF VIRAL INFECTIONS USING SPLIT ENZYMES

(71) Applicant: Brigham Young University, Provo, UT (US)

(72) Inventor: Jonathon T. HILL, Provo, UT (US)

(21) Appl. No.: 17/457,594

(22) Filed: Dec. 3, 2021

Related U.S. Application Data
(60) Provisional application No. 63/260,247, filed on Aug. 13, 2021.
(60) Provisional application No. 63/121,784, filed on Dec. 4, 2020.

(51) Int. Cl. G01N 33/569; G01N 33/58

(52) U.S. Cl. CPC G01N 33/56983; G01N 33/581; G01N 2333/908; G01N 2333/165; G01N 2470/04

(57) ABSTRACT

The composition includes a first construct having a first portion of a protein and a first antigen-recognizing amino acid sequence; and a second construct having a second portion of the protein that catalyzes a reaction when combined with the first portion of the protein and a second antigen-recognizing amino acid sequence. The first and second synthetic constructs include a sulfhydryl group configured such that a disulfide bond is formed between the first and second synthetic constructs when the first antigen-recognizing amino acid sequence and the second antigen-recognizing amino acid sequence bind an antigen.

### CROSS-REFERENCE TO RELATED APPLICATIONS

This application is a U.S. Non-provisional application claiming priority under 35 U.S.C. 120 and 119(e) to U.S. provisional application No. 63/121,784, filed Dec. 4, 2020 and to U.S. provisional application No. 63/260,247, filed Aug. 13, 2021, both of which are incorporated by reference in their entirety.

### REFERENCE TO SEQUENCE LISTING

A sequence listing entitled “Split_enzyme_349839. txt” is an ASCII text file and is incorporated herein by reference in its entirety. The text file was created on Feb. 10, 2022 and is 22.7 KB in size.

### BACKGROUND

### 1. Field of the Invention

The rapid, inexpensive, and sensitive detection of analytes in biological or environmental samples would greatly improve health and safety. For example, the detection of viral or bacterial infections and physiological biomarkers at home or the point of care would make diagnosis more accurate, rapid, and accessible while limiting undesirable exposure to others. Rapid and inexpensive tests would also allow frequent testing of water supplies, restaurant surfaces, and other potential sources of exposure to further decrease the spread of existing or emerging diseases.

Current methods, however, are limited due to the time, expertise, and often special equipment they require, leading to high costs and slow turnaround times. For example, the majority of current procedures for viral detection detect the genome of the virus. This, however, can be difficult as the genome must first be extracted from the viral capsid and, in some cases, converted from RNA to DNA before it can be amplified and detected. These procedures also require the production of several recombinant enzymes, which increases cost and may require significant hands-on time to process thereby delaying results.

Alternatively, antigenic tests have been developed to detect proteins on or in the viral capsid. These methods can detect intact virus, but are still labor intensive, slow, and costly. For example, an ELISA based assay typically requires three full antibodies, including a capture antibody, a detection antibody, and a secondary antibody. These assays also require multiple incubation and wash steps, decreasing throughput while increasing their cost and complexity. Therefore, a rapid test simple enough to be conducted by untrained personnel would greatly improve the availability and efficacy of viral testing measures, aiding both individual treatment and public health decisions.

The COVID-19 pandemic has highlighted the need for simple and rapid testing methods to accurately diagnose specific viral strains in individuals, even when they are asymptomatic or exhibit mild symptoms. The lack of availability of reliable rapid tests that could be quickly adapted to detect COVID-19 has led to increased viral spread, longer quarantine times, and broad lockdown measures. The severity of these measures has, in turn, decreased compliance and created resistance to continued efforts to control spread of the contagion. Beyond the current pandemic, a lack of a specific differential diagnoses methods in healthcare capable of discriminating between the common cold and influenza delays the use of antivirals, which are most effective when taken early in the course of the disease. Thus, the development of a simple, broadly applicable method to test for specific viral strains would improve individual treatment decisions and broad pandemic responses.

One proposed mechanism to simplify these assays is to create a split enzyme or multimeric protein complex that is reconstituted upon analyte binding. These methods, however, currently have limited sensitivity due the need for continuous binding to the analyte for enzymatic activity—preventing direct signal amplification. Thus, these systems must be designed to favor enzyme formation as much as possible to ensure sensitivity, but this can lead to the non-specific reconstitution of the enzyme in the absence of the analyte, resulting in a high number of false positives. To address these issues, complex mechanisms to separate the solution into various components or preprocessing steps are necessary, but this adds cost and complexity to the system. As a result, such systems have been limited to proximity labeling in cells, where the location of the enzyme can be tightly controlled and the samples processed before detection.

Similar advantages can be obtained by the simple and rapid detection of bacterial infections and other biological analytes. For example, the rapid detection at home or at the point of care of Streptococcus pyogenes infections could reduce burdens on the healthcare system and expedite treatment. In addition, the rapid and simple detection of insulin levels in blood or saliva samples could allow early detection of diabetes, improving health of millions of people worldwide.

### BRIEF SUMMARY

A composition is provided. The composition includes a first construct comprising a first portion of a protein and a first antigen-recognizing amino acid sequence; and a second construct comprising a second portion of the protein that catalyzes a reaction or is otherwise detectable when combined with the first portion of the protein and a second antigen-recognizing amino acid sequence. The first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

A method of detecting an analyte is also provided. The method includes adding a first construct comprising a first portion of a protein and a first antigen-recognizing amino acid sequence to a solution; adding a second construct to the solution, the second construct comprising a second portion of the protein that catalyzes a reaction or is otherwise detectable when combined with the first portion of the protein and a second antigen-recognizing amino acid sequence; and optionally adding a substrate to the solution. The first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

A kit is provided. The kit includes a first construct comprising a first portion of a protein and a first antigen-recognizing amino acid sequence; and a second construct comprising a second portion of the protein that catalyzes a reaction or is otherwise detectable when combined with the first portion of the protein and a second antigen-recognizing amino acid sequence. The first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

The foregoing broadly outlines the features and technical advantages of the present disclosure in order that the detailed description that follows may be better understood. Additional features and advantages of the disclosure will be described hereinafter that form the subject of the claims of this application. It will be appreciated by those of skill in the art that the conception and specific aspects disclosed herein may be readily utilized as a basis for modifying or designing other aspects for carrying out the same purposes of the present disclosure within the spirit and scope of the disclosure and provided in the appended claims.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

A detailed description of the invention is hereafter provided with specific reference being made to the drawings in which:

FIG. 1A shows workflow of the method of enzymatic viral detection.

FIG. 1B shows a graphical depiction of an embodiment showing the juxtaposition of the two enzyme segments by simultaneous binding to the viral surface protein, formation of the disulfide bond, and subsequent substrate conversion.

FIG. 2A: shows a schematic diagram of an embodiment of the corona-B38-HRPa and corona-H4-HRPb constructs. Each construct contains a portion of the HRP enzyme and the heavy (Vh) and light (Vl) chains from antibodies recognizing the COVID-19 spike protein. An N-terminal 6×His tag was also added to aid purification and testing.

FIG. 2B shows Dot Blot testing of the method using the fragments alone (Negative Control), an Anti-His antibody to link the two constructs by their epitope tags (Positive Control), and recombinant COVID-19 spike protein. Dark colors indicate positive detection.

FIG. 2C shows Dot Blot testing with varying concentrations of COVID-19 spike protein.

### DETAILED DESCRIPTION

Various aspects are described below with reference to the drawings. The relationship and functioning of the various elements of the aspects may better be understood by reference to the following detailed description. However, aspects are not limited to those illustrated in the drawings or explicitly described below. It should be understood that the drawings are not necessarily to scale, and in certain instances, details may have been omitted that are not necessary for an understanding of aspects disclosed herein, such as conventional fabrication and assembly.

To address the limitations of detecting analyte in a cell-free environment using split enzymes, a system is provided that regulates dimerization to favor reconstitution of the enzyme only when bound to the analyte. In addition, the disclosed compositions and methods provide for amplification of the detection signal Amplification can occur without adding an amplifier enzyme or reagent because once the constructs disclosed herein assemble on the target analyte, they can dissociate from the target analyte but will not disassemble and can continue to serve as a catalyst for signal production.

A composition is provided. The composition includes a first construct comprising a first portion of a protein and a first antigen-recognizing amino acid sequence; and a second construct comprising a second portion of the protein that catalyzes a reaction or is otherwise detectable when combined with the first portion of the protein and a second antigen-recognizing amino acid sequence. The first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

In some aspects, the first antigen-recognizing amino acid sequence and the second antigen-recognizing amino acid sequence each recognize different epitopes on a viral surface.

The constructs described here is also easily adaptable to different analytes. In some aspects, antibody fragments can be incorporated into the constructs to recognize the analyte of interest. For existing and emerging diseases, suitable antibodies can be easily identified because patients infected with the virus will develop antibodies to the viral surface as part of their normal immune response. These antibodies can be isolated and sequenced to design versions of this test for that viral isolate. In addition, several molecular techniques can be used, including phage display or selex technologies, to synthetically create highly efficient antibody fragments in the lab, providing multiple methods to adapt the technology to current and future diseases and other analytes.

“Antigen” refers to any protein, peptide, lipid, nucleic acid, carbohydrate, other chemical, or assembly thereof having at least two distinct epitopes to which an antibody can bind. In some aspects, the antigen comprises the viral spike protein of SARS-CoV-2.

The term “antibody”, also known as immunoglobulin (Ig), as used herein can be monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. The antibodies can be from any animal origin. An antibody can be IgG (including IgG1, IgG2, IgG3, and IgG4), IgA (including IgA1 and IgA2), IgD, IgE, or IgM, and IgY. In some embodiments, the antibody can be whole antibodies, including single-chain whole antibodies. In some embodiments, the antibody can be a fragment of an antibody, which can include, but are not limited to, a Fab, a Fab′, a F(ab′)2, an Fd (consisting of VH and CH1), an Fv fragment (consisting of VH and VL), a single-chain variable fragment (scFv), a single-chain antibody, a disulfide-linked variable fragment (dsFv), and fragments comprising either a VL or VH domain. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved.

The term “antigen-recognizing amino acid sequence” or its grammatical equivalents are used herein to mean one or more fragments or portions of an antibody that retain the ability to specifically bind to an antigen. In some embodiments, the antigen-recognizing amino acid sequence is a single chain Fv (scFv), which is a monovalent molecule consisting of the two domains of the Fv fragment (i.e., VL and VH) joined by a linker which enables the two domains to be synthesized as a single polypeptide chain

“Antigen recognition moiety,” “antigen recognition domain,” “antigen binding domain,” or “antigen binding region” refers to a molecule or portion of a molecule that specifically binds to an antigen. In one embodiment, the antigen recognition moiety is an antibody, antibody like molecule or fragment thereof.

In some aspects, the first antigen-recognizing amino acid sequence comprises at least one single-chain fragment variable (scFv).

In some aspects, the second antigen-recognizing amino acid sequence comprises at least one single-chain fragment variable (scFv).

In some aspects, the first antigen-recognizing amino acid sequence comprises at least one Fab fragment.

In some aspects, the second antigen-recognizing amino acid sequence comprises at least one Fab fragment.

In some aspects, the first antigen-recognizing amino acid sequence comprises at least one antibody.

In some aspects, the second antigen-recognizing amino acid sequence comprises at least one antibody.

In some aspects, the first construct further comprises an epitope tag for purification.

In some aspects, the second construct further comprises an epitope tag for purification.

In some aspects, the epitope tag is selected from the group consisting of: His, Flag, V5, Myc, HA, and epitope tags.

In some aspects, the first construct is SEQ ID NO: 1, SEQ ID NO: 3, or SEQ ID NO: 5. In some aspects, the second construct is SEQ ID NO: 2, SEQ ID NO: 4, or SEQ ID NO: 6. In some aspects, the first construct is SEQ ID NO: 1 and the second construct is SEQ ID NO: 2. In some aspects, the first construct is SEQ ID NO: 3 and the second construct is SEQ ID NO: 4. In some aspects, the first construct is SEQ ID NO: 5 and the second construct is SEQ ID NO: 6.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms. According to such analyses, groups of amino acids can be defined where amino acids within a group exchange preferentially with each other and, therefore, resemble each other most in their impact on the overall protein structure. Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH2 can be maintained. Exemplary conservative amino acid substitutions are shown in the following table:

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

In some aspects, the first construct is SEQ ID NO: 1, SEQ ID NO: 3, or SEQ ID NO: 5 or a conservatively substituted amino acid sequence thereof. In some aspects, the second construct is SEQ ID NO: 2, SEQ ID NO: 4, SEQ ID NO: 6, or a conservatively substituted amino acid sequence thereof. In some aspects, the first construct is SEQ ID NO: 1 or a conservatively substituted amino acid sequence thereof and the second construct is SEQ ID NO: 2 or a conservatively substituted amino acid sequence thereof. In some aspects, the first construct is SEQ ID NO: 3 or a conservatively substituted amino acid sequence thereof and the second construct is SEQ ID NO: 4 or a conservatively substituted amino acid sequence thereof. In some aspects, the first construct is SEQ ID NO: 5 or a conservatively substituted amino acid sequence thereof and the second construct is SEQ ID NO: 6 or a conservatively substituted amino acid sequence thereof.

In some aspects, the first construct comprises an amino acid sequence having at least 60%, at least 65%, at least 70%, at least 75%, at least 80%, at least 85%, at least 90%, at least 95%, at least 96%, at least 97%, at least 98%, or at least 99%, at least 99.5%, at least 99.9%, or 100% identity with any one of SEQ ID NOs: 1, 3, or 5.

In some aspects, the second construct comprises an amino acid sequence having at least 60%, at least 65%, at least 70%, at least 75%, at least 80%, at least 85%, at least 90%, at least 95%, at least 96%, at least 97%, at least 98%, or at least 99%, at least 99.5%, at least 99.9%, or 100% identity with any one of SEQ ID NOs: 2, 4, or 6.

The terms “identical” and its grammatical equivalents as used herein or “sequence identity” in the context of two amino acid sequences of polypeptides refer to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Alignment is also often performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. When one molecule is said to have certain percentage of sequence identity with a larger molecule, it means that when the two molecules are optimally aligned, the percentage of residues in the smaller molecule finds a match residue in the larger molecule in accordance with the order by which the two molecules are optimally aligned.

In some aspects, the first and second synthetic constructs comprise a sulfhydryl group configured such that a disulfide bond is formed between the first and second synthetic constructs. In some aspects, the first and second synthetic constructs comprise cysteines configured such that a disulfide bond is formed between the first and second synthetic constructs.

The protein attached to the antigen-recognizing amino acid sequences can be any one of two enzymatic subunits. Examples of enzymes include, but are not limited to, horseradish peroxidase, ascorbate peroxidase 2 (APEX2), Luciferase, and green fluorescent protein (GFP). In some aspects, the protein is GFP. In some aspects, the protein is horseradish peroxidase. In some aspects, the protein is APEX2. In some aspects, the protein is Luciferase.

The constructs described herein are expected to be very inexpensive to produce and distribute. Both the first and second constructs can be synthesized in standard E. coli bioreactors using standard expression methods. Other methods can be employed to produce the first and second constructs described herein such as expressing the polypeptides in other bacterial strains, yeast or other single-cell eukaryotes, mammalian cell lines (CHO cells, for example). In some aspects, the first and second constructs can be synthesized using in vitro or chemical synthesis techniques.

A method of detecting an analyte is also provided. The method includes adding a first construct comprising a first portion of a protein and a first antigen-recognizing amino acid sequence to a solution; adding a second construct to the solution, the second construct comprising a second portion of the protein that catalyzes a reaction or is otherwise detectable when combined with the first portion of the protein and a second antigen-recognizing amino acid sequence; and optionally adding a substrate to the solution.

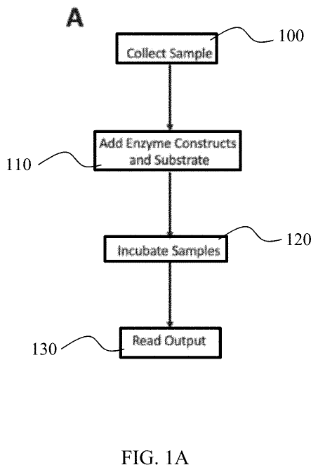

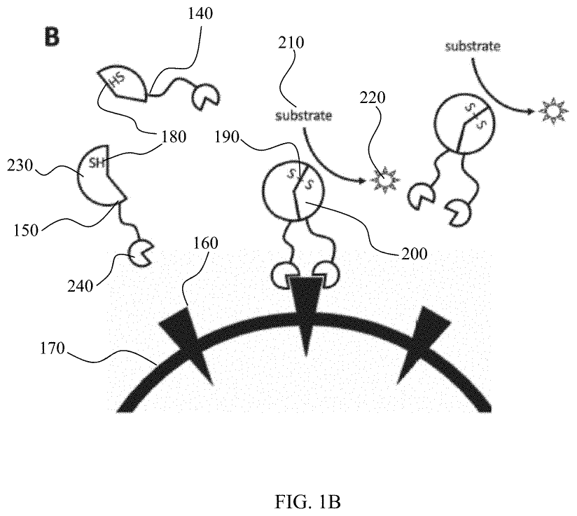

An embodiment of this simple and inexpensive method for rapid viral antigen detection is depicted in FIG. 1A. In some aspects, the method includes collecting a sample 100, adding the constructs and substrate 110, incubating the sample with the constructs and substrate 120, and reading the output 130.

FIG. 1B shows a first construct 140 and a second construct 150 before binding to an epitope 160 on the virus surface 170. In this aspect, each construct 140, 150 include a cysteine residue 180 capable of forming a disulfide bond 190 when the constructs 140, 150 bind to form a whole enzyme 200. The enzyme 200 can catalyze conversion of the substrate 210 into a detectable product 220. Each construct 140, 150 includes a portion of a protein 230 and an antigen-recognizing amino acid sequence 240.

In some aspects, two synthetic protein constructs incorporating portions of an oxidative enzyme were created. In the first construct, a first portion of the enzyme is linked to an antibody or antibody fragment recognizing an epitope on a viral surface protein. The second construct contains the complimentary fragment (second portion) of the enzyme linked to an antibody or antibody fragment that recognizes another epitope on the same viral protein or an adjacent protein. The two proteins are configured such that binding to the analyte brings the two enzyme segments into close proximity, allowing the full enzyme to be reconstituted and become active. When bound in an environment with a sufficiently oxidative redox potential, the enzyme fragments also form one or more disulfide bonds, stabilizing the functional enzyme. Because disulfide bonds only form when two cysteines are held in close proximity, this bond is unlikely to form in solution and will only form when held in place when the antibody or antibody fragments on the first and second construct are bound to their respective epitopes on the analyte. Reconstitution of the full enzyme allows it to catalyze a reaction or become otherwise detectable that can be measured to determine the presence of the analyte in the sample. The entire process can occur in a single tube containing the sample, resulting in a rapid and simple process for analyte detection that can be administered at the point of care or even at home.

This process is expected to be highly specific and sensitive. In some aspects, the specificity of the assay can be enhanced by the use of two distinct epitopes on the analyte. For example, many particularly dangerous viruses are closely related to much more innocuous strains. For example, the COVID-19 virus is closely related to other coronavirus strains that only produce mild cold-like symptoms. These close relationships can create false positive diagnoses in assays unable to distinguish between two. Using two epitopes unique to a particular viral strain is much less likely to cross react with other species than a single epitope.

Viral detection sensitivity is improved by three sources of amplification in this method. First, although a pathogen contains only one copy of its genome, many proteins repeat in a regular pattern on the surface, typically resulting in many copies of surface proteins per genome. The second amplification occurs as a result of the formation of the disulfide bond between the enzyme portions. Antibody binding is a transient process and is typically too weak to detect protein levels at low analyte concentrations. By designing the system to create disulfide bonds upon binding, however, the reconstituted enzyme will remain active even after release from the analyte. Release will also free the antigen for another antibody fragment to bind, allowing more enzymes to be made per analyte in solution than methods, such as ELISA assays, that require continual binding to the analyte. Finally, amplification occurs during enzymatic catalysis of the substrate to create a measurable product, further strengthening the signal to allow detection of very low amounts of material in the sample.

For each test, several parameters in the assay can also be tuned to control the threshold of detection. For example, it is commonly known that viral detection assays that are not sufficiently sensitive may create false negatives, greatly undermining the value of the test. Overly sensitive tests, however, are also a concern. One criticism of current qPCR-based viral detection methods is that a few genome copies arising from dead viral particles can be sufficient to trigger a positive result, even though no live virus is in the sample and the patient is not at risk of spreading infection. This can lead to unnecessary treatment or quarantine. In this disclosure, the amount of enzyme formed from construct association, the amount of substrate, the length of incubation time, the affinity of the antibody fragments (including the addition of multiple antibody fragments on each synthetic protein), buffer conditions, and the inclusion of preprocessing procedures to concentrate or purify the sample can be tuned to control the limit of detection to a reasonable level. Together, its adaptability and tunability will create a broadly applicable and informative testing method.

In some aspects, the substrate is 3,3′,5,5′-tetramethylbenzidine, 5-amino-2,3-dihydrophthalazine-1,4-dione, or luciferin. In some aspects, the substrate is 3,3′,5,5′-tetramethylbenzidine. In some aspects, the substrate is 5-amino-2,3-dihydrophthalazine-1,4-dione. In some aspects the substrate is luciferin.

In some aspects, the substrate is luminol, 2,2′-Azinobis [3-ethylbenzothiazoline-6-sulfonic acid]-diammonium salt (ABTS), 3-Amino-9-ethylcarbazole (AEC), 3,3′Diaminobenzidine (DAB), enhanced chemiluminescence (ECL), o-phenylenediamine dihydrochloride (OPD), or Amplex Red.

In some aspects, the solution comprises a buffer that creates an oxidative environment. Examples of suitable buffers include, but are not limited to, a 0.05 M Phosphate-Citrate Buffer, containing 0.001-0.01% H₂O₂ or 0.03% sodium perborate having a pH of 5.0 or 1M Tris-HCl, containing 0.001-0.01% H₂O₂ having a pH of 8.5. In some aspects, the buffers may further include an enhancer such as p-Coumeric Acid; 4-iodophenylboronic acid (4IPBA); 4-tert-butylphenol; or p-cresol.

In addition to changing the amino acid sequence, several buffer parameters, including ionic strength, pH, temperature, presence of detergents and blocking proteins, and the overall concentration of the two constructs, can be optimized to control dimer formation rate. Thus, this system is tunable to a level not previously seen in current proximity labeling or analyte detection methods. Together, these optimizations will allow each assay based on this technology to carefully control sensitivity, specificity, reaction time, and other aspects of detection.

In some aspects, the solution comprises a sample collected from a subject.

In some aspects, the subject is human.

In some aspects, the sample comprises saliva, mucous, blood, urine, feces, or combinations thereof.

A kit is provided. The kit includes a first construct comprising a first portion of a protein and a first antigen-recognizing amino acid sequence; and a second construct comprising a second portion of the protein that catalyzes a reaction or is otherwise detectable when combined with the first portion of the protein and a second antigen-recognizing amino acid sequence.

In some aspects, the kit includes written materials e.g., instructions for use of the constructs, substrate, and solutions. Without limitation, the kit may include buffers, diluents, filters, needles, syringes, and package inserts with instructions for performing any methods disclosed herein.

The following examples provide and illustrate certain features and/or aspects of the disclosure. The examples should not be construed to limit the disclosure to the particular features or aspects described therein.

### EXAMPLES

### Example 1

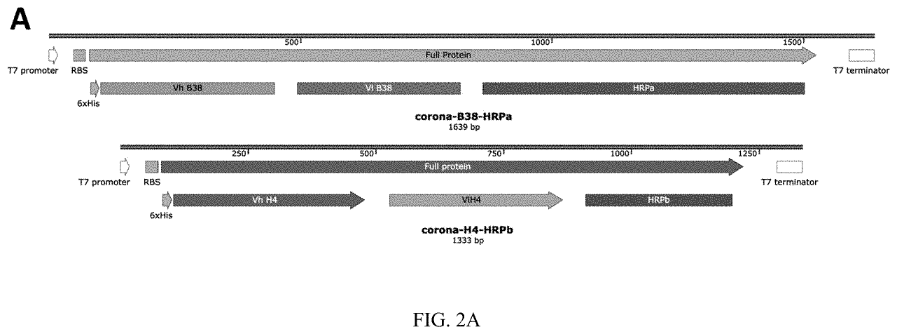

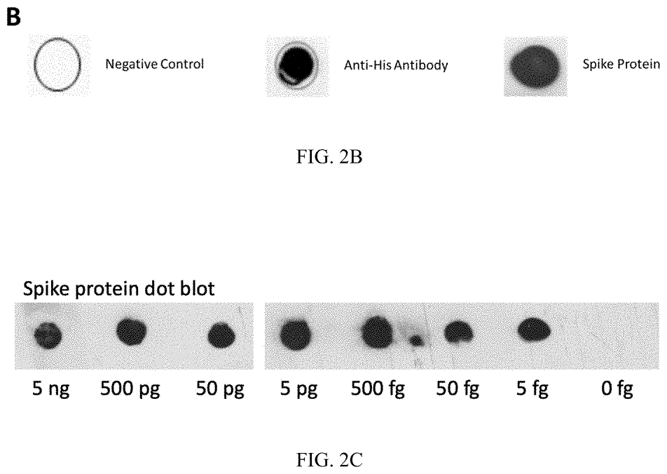

First, two synthetic proteins were designed and produced to incorporate portions of an HRP enzyme (HRPa and HRPb) previously shown to reconstitute an active enzyme when expressed on the surface of two touching cells. Each HRP portion was linked to an scFv designed from a pair of antibodies isolated from an early COVID-19 patient and shown to noncompetitively bind two epitopes on the viral spike protein. Finally, a 6×His epitope tag was affixed to aid purification and testing of the protein products (FIG. 2A). The proteins were expressed separately in a standard E. coli expression strain (SHUFFLE) and purified using a nickel chromatography column using standard procedures. The appropriate amount of spike protein (R&D Systems) was then mixed with 1 ug of each HRP fragment and incubated at room temperature for 15 minutes. The reaction was then blotted onto a nitrocellulose membrane and allowed to dry. The dried membrane was rewet with TBST and blocked using 5% milk for 30 minutes. The blot was then rinsed and luminol reagent/enhancer (Bio-rad) was added according to the manufacturer's instructions. The blots were then exposed to film for 30 seconds to 1 minute and developed.

The dot blot analysis containing either the synthetic polypeptides alone (negative control), an anti-His antibody to link the two polypeptides via their epitope tags (positive control), or 500 ng of recombinant COVID-19 spike protein showed that the method was effective, even without optimization (FIG. 2B). A preliminary limit-of-detection test was conducted as described above, except varying concentrations of spike protein from 5 fg to 5 ng were used (FIG. 2C). Ongoing tests are being conducted to optimize the method.

### Statements

Statement 1: A composition for analyte detection, comprising: a first construct comprising a first portion of a protein and a first antigen-recognizing amino acid sequence; and a second construct comprising a second portion of the protein that catalyzes a reaction when combined with the first portion of the protein and a second antigen-recognizing amino acid sequence, wherein the first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

Statement 2: The composition of statement 1, wherein the first antigen-recognizing amino acid sequence and the second antigen-recognizing amino acid sequence each recognize different epitopes on a viral surface.

Statement 3: The composition of any of statements 1 to 2, wherein the protein is horseradish peroxidase.

Statement 4: The composition of any of statements 1 to 3, wherein the first antigen-recognizing amino acid sequence comprises at least one single-chain fragment variable (scFv).

Statement 5: The composition of any of statements 1 to 4, wherein the second antigen-recognizing amino acid sequence comprises at least one single-chain fragment variable (scFv).

Statement 6: The composition of any of statements 1 to 3, wherein the first antigen-recognizing amino acid sequence comprises at least one Fab fragment.

Statement 7: The composition of any of statements 1 to 3 and 6, wherein the second antigen-recognizing amino acid sequence comprises at least one Fab fragment.

Statement 8: The composition of any of statements 1 to 3, wherein the first antigen-recognizing amino acid sequence comprises at least one antibody.

Statement 9: The composition of any of statements 1 to 3 and 8, wherein the second antigen-recognizing amino acid sequence comprises at least one antibody.

Statement 10. The composition of any of statements 1 to 9, wherein the first construct further comprises an epitope tag for purification.

Statement 11: The composition of any one of statements 1 to 9, wherein the second construct further comprises an epitope tag for purification.

Statement 12: The composition of any one of statements 10 to 11, wherein the epitope tag is selected from the group consisting of: His, Flag, V5, Myc, HA, and epitope tags.

Statement 13: The composition of any one of statements 1 to 12, wherein the protein is horseradish peroxidase, ascorbate peroxidase 2 (APEX2), Luciferase, or green fluorescent protein (GFP).

Statement 14: A method of detecting an analyte, comprising: adding a first construct comprising a first portion of a protein and a first antigen-recognizing amino acid sequence to a solution; adding a second construct to the solution, the second construct comprising a second portion of the protein that catalyzes an oxidative reaction when combined with the first portion of the protein and a second antigen-recognizing amino acid sequence; and adding a substrate to the solution.

Statement 15: The method of statement 14, wherein the substrate is 3,3′,5,5′-tetramethylbenzidine or 5-amino-2,3-dihydrophthalazine-1,4-dione.

Statement 16: The method of any one of statements 14 to 15, wherein the solution comprises a buffer that creates an oxidative environment.

Statement 17: The method of any one of statements 14 to 16, wherein the solution comprises a sample collected from a subject.

Statement 18: The method of statement 17, wherein the subject is human.

Statement 19: The method of any one of statements 17 to 18, wherein the sample comprises saliva, mucous, blood, urine, feces, or combinations thereof.

What is claimed is:

1. A composition for analyte detection, comprising:
a first construct comprising a first portion of a protein and a first antigen-recognizing amino acid sequence; and
a second construct comprising a second portion of the protein that catalyzes a reaction when combined with the first portion of the protein and a second antigen-recognizing amino acid sequence,
wherein the first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

2. The composition of claim 1, wherein the first antigen-recognizing amino acid sequence and the second antigen-recognizing amino acid sequence each recognize different epitopes on a viral surface.

3. The composition of claim 1, wherein the protein is horseradish peroxidase.

4. The composition of claim 1, wherein the first antigen-recognizing amino acid sequence comprises at least one single-chain fragment variable (scFv).

5. The composition of claim 1, wherein the second antigen-recognizing amino acid sequence comprises at least one single-chain fragment variable (scFv).

6. The composition of claim 1, wherein the first antigen-recognizing amino acid sequence comprises at least one Fab fragment.

7. The composition of claim 1, wherein the second antigen-recognizing amino acid sequence comprises at least one Fab fragment.

8. The composition of claim 1, wherein the first antigen-recognizing amino acid sequence comprises at least one antibody.

9. The composition of claim 1, wherein the second antigen-recognizing amino acid sequence comprises at least one antibody.

10. The composition of claim 1, wherein the first construct further comprises an epitope tag for purification.

11. The composition of claim 1, wherein the second construct further comprises an epitope tag for purification.

12. The composition of claim 10, wherein the epitope tag is selected from the group consisting of: His, Flag, V5, Myc, HA, and epitope tags.

13. The composition of claim 1, wherein the protein is horseradish peroxidase, ascorbate peroxidase 2 (APEX2), Luciferase, or green fluorescent protein (GFP).

14. A method of detecting an analyte, comprising:
adding a first construct comprising a first portion of a protein and a first antigen-recognizing amino acid sequence to a solution;
adding a second construct to the solution, the second construct comprising a second portion of the protein that catalyzes an oxidative reaction when combined with the first portion of the protein and a second antigen-recognizing amino acid sequence; and
optionally adding a substrate to the solution,
wherein the first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.

15. The method of claim 14, wherein the substrate is 3,3′,5,5′-tetramethylbenzidine or 5-amino-2,3-dihydrophthalazine-1,4-dione.

16. The method of claim 14, wherein the solution comprises a buffer that creates an oxidative environment.

17. The method of claim 14, wherein the solution comprises a sample collected from a subject.

18. The method of claim 17, wherein the subject is human.

19. The method of claim 17, wherein the sample comprises saliva, mucous, blood, urine, feces, or combinations thereof.

20. A kit, comprising:
a first construct comprising a first portion of a protein and a first antigen-recognizing amino acid sequence; and
a second construct to the solution, the second construct comprising a second portion of the protein that catalyzes a reaction when combined with the first portion of the protein and a second antigen-recognizing amino acid sequence,
wherein the first and second synthetic constructs comprise a sulfhydryl group configured such that a disulfide bond is formed between the first and second synthetic constructs when the first antigen-recognizing amino acid sequence and the second antigen-recognizing amino acid sequence bind an antigen.


SEQUENCE LISTING (6 sequences)

SEQ ID NO: 1 - protein, 481 aa, Artificial Sequence
  Split HRP 1 with COVID19 scfv
MHHHHHHMEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGSGGGGSGGGGSQLTPTFYDNSCPNVSNIVRDIIVNELRSDPRIAASILRLHFHDCFVNGCDASILLDNTTSFRTEKDAFGNANSARGFSVIDRMKAAVESACPGTVSCADLLTIAAQQSVTLAGGPSWRVPLGRRDSLQAFLDLANANLPAPFFTLPQLKDSFRNVGLNRSSDLVALSGGHTFGKSQCRFIMDRLYNFSNTGLPDPTLNTTYLQTLRGLCPLNGGSGKDEL

SEQ ID NO: 2 - protein, 379 aa, Artificial Sequence
  Split HRP 2 with COVID19 scfv
MHHHHHHMQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSSGGGGSGGGGSGGGGSDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKGGGGSGGGGSGGGGSNLSALVDFDLRTPTIFDNKYYVNLEEQKGLIQSDQELFSSPDATDTIPLVRSFANSTQTFFNAFVEAMDRMGNITPLTGTQGQIRRNCRVVNSNSGSGKDEL

SEQ ID NO: 3 - protein, 534 aa, Artificial Sequence
  Split Apex 1 with COVID19 scfv
MGLNDIFEAQKIEWHEGGGGSMGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSCSGGGGSGGGGSGGGGSGGGGSGGGGSGGGGSMDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKSGGGGSGGGGSGGGGSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSGGGGSHHHHHH

SEQ ID NO: 4 - protein, 362 aa, Artificial Sequence
  Split Apex 2 with COVID19 scfv
MHHHHHHGGGGSMEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGSGGGGSGGGGSGGGGSGGGGSGGGGSCSGGGGSGGGGSGGGGSASGLLQLPSDKALLSDPVFRPLVDKYAADEDAFFADYAEAHQKLSELGFADALQLPPLERLTLD

SEQ ID NO: 5 - protein, 364 aa, Artificial Sequence
  Split GFP 1 with COVID19 scfv
MHHHHHHMEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKSGGGGSGGGGSGGGGSGGGGSGGGGSGGGGSCSGGSGKNGIKVNFKTRHNIEDGSVQLADHYQQNTPIGDGPVLLPDNHYLSTQSALSKDPNEKRDHMVLLEFVTAAGITHGMDELYN

SEQ ID NO: 6 - protein, 483 aa, Artificial Sequence
  Split GFP 2 with COVID19 scfv
MGLNDIFEAQKIEWHEGGGGSMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNHNVLIMADKQGGSGSGCSGGGGSGGGGSGGGGSGGGGSGGGGSGGGGSGMQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSSGGGGSGGGGSGGGGSDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKSGGGSHHHHHH